(19) United States
(12) Patent Application Publication
(10) Pub. No.: US 2022/0288009 A1
(43) Pub. Date: Sep. 15, 2022

(54) CABAZITAXEL LIQUID FORMULATIONS

(71) Applicant: SHILPA MEDICARE LIMITED, RAICHUR (IN)

(72) Inventors: KIRAN KRISHNAPPA JADHAV, Bangalore (IN); PRASHANTH SHANKAR, Bangalore (IN); PRADEEP SHIVAKUMAR, Vizianagaram (IN); SREENIVASA REDDY, Bangalore (IN)

(73) Assignee: SHILPA MEDICARE LTD, RAICHUR (IN)

(21) Appl. No.: 17/633,964

(22) PCT Filed: Sep. 3, 2020
(86) PCT No.: PCT/IB2020/058196

§ 371 Date: Feb. 9, 2022

(30) Foreign Application Priority Data
Sep. 6, 2019 (IN) 201941035968

(51) Int. Cl. A61K 31/337; A61K 47/24; A61K 47/28; A61K 47/32; A61K 47/10; A61K 9/08

(52) U.S. Cl. CPC A61K 31/337; A61K 47/24; A61K 47/28; A61K 47/32; A61K 47/10; A61K 9/08

(57) ABSTRACT

The present invention relates to the stable liquid pharmaceutical composition comprising (a) cabazitaxel or a pharmaceutically acceptable salt or solvate thereof, (b) one or more solubilizers selected from the group consisting of soya phosphatidyl choline, polyethylene glycol and glycocholic acid, (c) a solvent and (d) a co-solvent, wherein the pharmaceutical composition is substantially free of (i) polysorbates and (ii) stabilizers selected from the group consisting of preservatives, antioxidants and chelating agents and the process for preparation thereof, and the methods of using the stable liquid pharmaceutical composition of cabazitaxel for the treatment of patients with hormone refractory metastatic prostate cancer previously treated with a docetaxel containing treatment regimen.

### FIELD OF THE INVENTION

The present invention relates to pharmaceutical formulations comprising cabazitaxel or a pharmaceutically acceptable salt or solvate thereof and a solubilizer selected from group comprising of phosphatidyl choline, polyethylene glycol, glycocholic acid and/or mixtures thereof, wherein the formulation is substantially free of polysorbates and stabilizers selected from the group consisting of preservatives, antioxidants and chelating agents.

The present invention also relates to pharmaceutical formulations comprising cabazitaxel or a pharmaceutically acceptable salt or solvate thereof, a solubilizer selected from group comprising of phosphatidyl choline, polyethylene glycol, glycocholic acid and/or mixtures thereof, solvent and a co-solvent.

In addition, the present invention relates to pharmaceutical formulations comprising cabazitaxel or a pharmaceutically acceptable salt or solvate thereof, a solubilizer selected from group comprising of phosphatidyl choline, polyethylene glycol, glycocholic acid and/or mixtures thereof, solvent and a co-solvent and an infusion solution in order to administer the formulation to patients.

In addition, the present invention relates to methods for administering the formulations to patients in need thereof and for preparing the formulations.

### BACKGROUND OF THE INVENTION

Cabazitaxel is an antineoplastic agent belonging to the taxane class with the chemical name (2α, 5β, 7β, 10β, 13α)-4-acetoxy-13-({(2R,3S)-3-[(tertbutoxycarbonyl) amino]-2-hydroxy-3-phenylpropanoyl}oxy)-1-hydroxy-7,10-dimethoxy-9-oxo-5,20-epoxytax-11-en-2-yl benzoate. Cabazitaxel is the 7,10-dimethoxy analogue of docetaxel and like other members of taxane family, it is also a microtubule inhibitor, which is presently approved worldwide, in combination with prednisone, for treatment of patients with hormone-refractory metastatic prostate cancer previously treated with a docetaxel-containing treatment regimen.

Cabazitaxel is marketed worldwide under the brand name of JEVTANA® by Sanofi Aventis. JEVTANA® is supplied as a kit consisting of (a) a JEVTANA® injection, which contains 60 mg cabazitaxel in 1.5 mL polysorbate 80; and (b) a diluent, containing approximately 5.7 mL 13% (w/w) ethanol. Prior to administration, the JEVTANA® injection must first be mixed with the diluent, which dilutes the amount of cabazitaxel to 10 mg/mL, and then further diluted into a 250 mL PVC-free container of either 0.9% sodium chloride solution or 5% dextrose solution for infusion. The concentration of cabazitaxel in the resulting final infusion solution should be between 0.10 mg/mL and 0.26 mg/mL.

The presence of polysorbate 80 in JEVTANA®, can result in serious side effects. Such reactions characterized by generalized rash/erythema, hypotension and/or bronchospasm, or very rarely fatal anaphylaxis, have been reported in patients. Hypersensitivity reactions may require immediate discontinuation of the taxane infusion and administration of appropriate therapy. In order to reduce the side effects induced by polysorbate 80, patients may be treated with dexamethasone prior to each dose of JEVTANA®. Dexamethasone is a steroid that suppresses the immune response in patients, which can be especially detrimental in cancer patients under chemotherapy, whose immunity may already be compromised due to the destruction of healthy cells by the chemotherapeutic treatment. As a result, these patients can be susceptible to bacterial and fungal infections. Further, despite receiving the dexamethasone premedication, patients can report hypersensitivity side effects from the taxane compound treatment. Due to these side effects, patients may stop cabazitaxel compound therapy, skip a dose, or continue further therapy at a reduced dose.

Further JEVTANA® injection is a micellar formulation. The pre-mix solution is prepared by first dilution in a supersaturated solution by about 400% and is inherently physically unstable. It requires repeated inversions for at least 45 seconds to assure complete mixing of the concentrated drug solution and the diluent. The pre-mix solution, having a concentration of 10 mg of cabazitaxel per mL should be used immediately, preferably within 30 minutes and requires further dilution before administration. Even after second dilution, the concentration of cabazitaxel in the solution remains supersaturated and therefore should be used for intravenous administration immediately, with 8 hours, if stored at room temperature or with 24 hours, if stored under refrigeration conditions. Further, these supersaturated solutions are prone to crystallization and hence the prescribing information for JEVTANA® instructs that if crystals and/or particulates appear in the diluted infusion solution, it must not be used and should be discarded.

PCT Publication No. WO2013024495A1 discloses a pharmaceutical formulation for parenteral administration comprising cabazitaxel or a pharmaceutically acceptable salt thereof and at least one solubilizer, wherein solubilizer is selected from group comprising polysorbates, polyethylene glycols, propylene glycol, tetraglycol, glycerol, ethanol and a mixture thereof and further the scope of invention of WO '495 Patent Application discloses the use of antioxidants and chelating agents to reduce the degradation of cabazitaxel or any other excipient present in the formulation.

PCT Publication No. WO2013022960A1 discloses a sterile pharmaceutical formulation comprising cabazitaxel which is substantially free of polysorbates and polyethoxylated castor oil. The composition comprises of cabazitaxel or a pharmaceutically acceptable salt thereof; a solubilizer, wherein solubilizer is selected from glycofurol and ethanol, tocopherol polyethylene glycol succinate (TPGS), one or more hydrotropes and optionally one or more agents having a pKa of about 3 to about 6 and optionally one or more antioxidizing agent.

PCT Publication No. WO2014028704A1 discloses an enclosed liquid pharmaceutical composition container, comprising a liquid phase and a gaseous phase, wherein the liquid phase comprises cabazitaxel, polysorbate 80, ethanol, and one or more pH adjusters to maintain pH about 2.8-6.0, and the gaseous phase is saturated with CO₂.

U.S. Pat. No. 10,278,946 discloses a pharmaceutical composition consisting of: cabazitaxel or a pharmaceutically acceptable salt thereof; a mixture of macrogol 15 hydroxy stearate and polyethylene glycol with a molecular weight range from 200 to 600; ethanol; and citric acid. Citric acid in U.S. '946 is an antioxidant, which is used to reduce the degradation of cabazitaxel in the pharmaceutical composition.

Therefore, new formulations of cabazitaxel or other taxane compounds are needed to avoid these side effects, premedication requirements, and patient noncompliance issues associated with the currently marketed formulation. Further there remains a need for a stable, single-vial formulation for cabazitaxel, which needs to be diluted only once for intravenous infusion, which are substantially free of polysorbates and stabilizers selected from the group consisting of preservatives, antioxidants and chelating agents. Ideally, such formulations would be conveniently prepared for use and would exhibit enhanced storage stability at ambient conditions.

### OBJECTS OF THE INVENTION

The first object of the invention is to provide a stable pharmaceutical composition comprising cabazitaxel or a pharmaceutically acceptable salt or solvate thereof.

Another object of the present invention is to provide a stable single-vial formulation, suitable for parenteral administration comprising cabazitaxel or a pharmaceutically acceptable salts or solvate thereof, which is ready for direct dilution with an infusion solution or for direct introduction into an infusion bag.

Another object of the present invention is to provide a stable single-vial formulation, suitable for parenteral administration with stable concentration of 10 mg/mL with 6 mL fill comprising cabazitaxel or a pharmaceutically acceptable salts or solvate thereof, which is ready for direct dilution with an infusion solution or for direct introduction into an infusion bag.

Yet another object of the present invention is to provide a stable injectable pharmaceutical formulation comprising cabazitaxel or a pharmaceutically acceptable salt or solvate thereof and a solubilizer selected from group comprising of soya phosphatidyl choline, polyethylene glycol, glycocholic acid and/or mixtures thereof; wherein the formulation is substantially free of polysorbates and stabilizers selected from group consisting of preservatives, antioxidants and chelating agents.

Further another object of the present invention is to provide the method of treating prostate cancer by administering a stable injectable pharmaceutical formulation comprising cabazitaxel or a pharmaceutically acceptable salt or solvate thereof, and a solubilizer selected from group comprising of soya phosphatidyl choline, polyethylene glycol, glycocholic acid and/or mixtures thereof, wherein the formulation is substantially free of polysorbates and stabilizers selected from group consisting of preservatives, antioxidants and chelating agents, and wherein the treatment does not require the administration of dexamethasone premedication.

Further another object of the present invention is to provide a simple, commercially viable process for preparation of an injectable formulation of cabazitaxel.

Further another object of the present invention is formation of nano dispersion when reconstituted with 0.9% sodium chloride and 5% dextrose.

### SUMMARY OF THE INVENTION

The present inventors have developed the sterile and stable liquid formulations of cabazitaxel or a pharmaceutically acceptable salt or solvates thereof, that is substantially free of polysorbates and stabilizers selected from group consisting of preservatives, antioxidants and chelating agents, and does not require the dexamethasone premedication for the treatment of prostate cancer. These cabazitaxel formulations are single-vial concentrates, with 10 mg/mL concentration which are sterile liquids in a single vial ready to be diluted with an infusion solution.

Thus, in one embodiment, the present invention provides a stable pharmaceutical formulation for use in treatment of a patient in need thereof, comprising cabazitaxel or a pharmaceutically acceptable salt or solvate thereof and a solubilizer, wherein solubilizer is selected from group comprising of soya phosphatidyl choline, polyethylene glycol and glycocholic acid.

In another embodiment, the present invention provides a stable pharmaceutical formulation for use in treatment of a patient in need thereof comprising:
a) cabazitaxel or a pharmaceutically acceptable salt or solvate thereof,
b) one or more solubilizer, wherein solubilizer is selected from group comprising of soya phosphatidyl choline, polyethylene glycol and glycocholic acid,
c) a solvent and
d) a co-solvent.

In yet another embodiment, the stable pharmaceutical formulation of present invention is a ready to dilute formulation, suitable for parenteral administration.

In a specific embodiment, the present invention provides a stable pharmaceutical formulation for use in treatment of a patients in need thereof comprising
a) cabazitaxel or a pharmaceutically acceptable salt or solvate thereof,
b) soya phosphatidyl choline,
c) polyethylene glycol,
d) glycocholic acid,
e) ethanol and
f) polyvinylpyrrolidone.

In a further embodiment, the present invention provides a stable pharmaceutical formulation for use in treatment of patients in need thereof comprising
a) cabazitaxel or a pharmaceutically acceptable salt or solvate thereof,
b) soya phosphatidyl choline,
c) polyethylene glycol,
d) ethanol and
e) polyvinylpyrrolidone.

### DETAILED DESCRIPTION OF THE INVENTION

The present invention is directed to a stable and sterile liquid pharmaceutical composition, comprising cabazitaxel or a pharmaceutically acceptable salt or solvate thereof, at least one solubilizer, wherein the pharmaceutical composition is substantially free of polysorbates and stabilizers selected from group consisting of preservatives, antioxidants and chelating agents.

The present invention relates to cabazitaxel formulations that is substantially free of polysorbates may be administered to patients without pre-medicating with steroids, wherein the steroid is preferably dexamethasone. The reduction or elimination of the steroid pre-treatment phase can reduce concerns of immune system depression and other side effects, as well as of interactions with other drugs that the patient may be taking. Also, eliminating polysorbates in the formulation can remove the risk of skin rashes, edema, hypotension and bradycardia.

The present invention is directed to stable liquid composition of cabazitaxel, presented as a single-vial injectable solution, ready for direct dilution with an infusion solution containing 5% dextrose solution or 0.9% sodium chloride solution, without need for preparation of premix solution.

As used here in “single-vial Injection Solution” refers to a sterile liquid in a single vial that can be administered by intravenous route to a patient upon dilution with only an infusion solution. i.e., no other dilution may be necessary before dilution with the infusion solution.

As used herein the term “nanoparticles” means any particle having controlled dimensions of the order of nanometers.

As used herein “final dilution for infusion” refers to the result of the single-vial injection solution combined with an infusion solution. The final dilution for infusion may be in the form of nanodispersion with the particle size less than 400 nm and may be ready to be administered to a patient.

As used herein, “substantially free” refers to presence of a material in an amount of less than about 0.01% w/v or about 0% w/v (i.e. totally free) in the stable liquid composition.

“Infusion solution” refers to a sterile isotonic solution, typically stored in a bag or bottle that is employed to dilute the single-vial injection concentrate or the diluted injection concentrate for administration to a patient.

As used herein, the term “cabazitaxel” includes the compound cabazitaxel, pharmaceutically acceptable salts of cabazitaxel, isomers, solvates, complexes and hydrates, anhydrous forms thereof, and any polymorphic or amorphous form or combinations thereof.

As used herein, the term “stable compositions” refers to any preparation of cabazitaxel having sufficient stability to allow storage at a convenient temperature, such as between about 0° C. and about 60° C. for a pharmaceutically acceptable duration of time. Preferably, the composition is stable for a period of time, such as at least about one week, at least about one month, at least about three months, at least about six months, at least about one year, or at least about 2 years.

As used herein, the term “solubilizer” refers to a solvent that is capable of dissolving cabazitaxel, or a pharmaceutically acceptable salt thereof.

The formulations of the present invention are particularly suited for use in parenteral administration which does not require the steroid premedication (dexamethasone) for treatment of patients with hormone-refractory metastatic prostate cancer.

In embodiments of the present invention, the present invention relates to a stable liquid pharmaceutical composition, comprising cabazitaxel or a pharmaceutically acceptable salt or solvate thereof, at least one solubilizer, solvent and a cosolvent.

In embodiments of the present invention, the present invention relates to stable liquid pharmaceutical composition, comprising cabazitaxel or a pharmaceutically acceptable salt or solvate thereof, at least one solubilizer, solvent and a cosolvent, wherein the pharmaceutical composition is substantially free of polysorbates and stabilizers selected from group consisting of preservatives, antioxidants and chelating agents.

In particular embodiments of the invention, the stable liquid pharmaceutical composition of cabazitaxel of the present invention is free of stabilizers selected from group consisting of preservatives, antioxidants and chelating agents. The stabilizers may be considered a preservative, antioxidant, chelating agents, yet, itself does not destabilize the formulation. Useful but exemplary antioxidants include one or more of cysteine, acetylcysteine, thioglycerol, butylated hydroxy toluene, butylated hydroxy anisole, citric acid, sodium metabisulfite, sodium thiosulfate, alpha tocopherol or a combination thereof, chelating agents (e.g., citrate, malic acid, edetate, or pentetate), sodium pyrophosphate, and sodium gluconate and preservatives (e.g., methyl paraben, propyl paraben, benzalkonium chloride).

In another preferred embodiment, the present invention provides a stable liquid pharmaceutical composition comprising cabazitaxel at concentrations about 5 mg/mL to about 200 mg/mL. Typically, the concentrations of cabazitaxel are in the range of about 10 mg/mL to about 100 mg/mL.

The solubilizers used in the present invention include, but are not limited to glycols, phospholipids, cholic acid, and glycocholic acid and/or mixtures thereof.

The glycol is preferably selected from the group consisting of polyethylene glycols, propylene glycol, tetra glycol and mixtures thereof. Polyethylene glycol (e.g. PEG 300 and PEG 400) is an excipient which is widely used in pharmaceutical formulations. Preferably, the polyethylene glycol has a molecular weight in the range from 200 to 600. More preferably, the polyethylene glycol has a molecular weight of about 400 (PEG 400).

Phospholipids is preferably selected from the group consisting of lecithin (egg yolk) based phospholipids, soya phosphatidylcholine (SPC) which include the following phosphatidylcholines, palmitoyloleoyl-phosphatidylcholine, palmitoyllinoleoylphosphatidylcholine, stearoyllinoleoylphosphatidyl-choline, stearoyloleoylphosphatidylcholine, stearoylarachidoylphosphatidylcholine, and dipalmitoylphosphatidylcholine. Other phospholipids including L-α-dimyristoyl-phosphatidylcholine (DMPC), dioleoylphosphatidylcholine (DOPC), distearoylphosphatidyl-choline (DSPC), hydrogenated soya phosphatidylcholine (HSPC), and other related compounds.

In one embodiment the present invention relates to a stable liquid pharmaceutical composition comprising
a) cabazitaxel or a pharmaceutically acceptable salt or solvate thereof, and
b) one or more solubilizers selected from the group consisting of soya phosphatidyl choline, polyethylene glycol and glycolic acid.

In a further embodiment the present invention relates to a stable liquid pharmaceutical composition comprising
a) cabazitaxel or a pharmaceutically acceptable salt or solvate thereof, and
b) one or more solubilizers selected from the group consisting of soya phosphatidyl choline, polyethylene glycol and glycocholic acid, wherein the pharmaceutical composition is substantially free of
   (i) polysorbates and
   (ii) stabilizers selected from the group consisting of preservatives, antioxidants and chelating agents.

In one embodiment the present invention relates to a stable liquid pharmaceutical composition comprising
a) about 5 mg/mL to about 200 mg/mL of cabazitaxel or a pharmaceutically acceptable salt or solvate thereof, and
b) one or more solubilizers selected from the group consisting of soya phosphatidyl choline, polyethylene glycol and glycocholic acid.

In another embodiment the present invention relates to a stable liquid pharmaceutical composition comprising
a) about 5 mg/mL to about 200 mg/mL of cabazitaxel or a pharmaceutically acceptable salt or solvate thereof, and
b) one or more solubilizers selected from the group consisting of soya phosphatidyl choline, polyethylene glycol and glycocholic acid, wherein the pharmaceutical composition is substantially free of
   (i) polysorbates and
   (ii) stabilizers selected from the group consisting of preservatives, antioxidants and chelating agents.

In a further embodiment, the present invention relates to a stable liquid pharmaceutical composition comprising
a) about 5 mg/mL to about 200 mg/mL of cabazitaxel or a pharmaceutically acceptable salt or solvate thereof, and
b) a solubilizer, wherein the solubilizer is a mixture of phosphatidyl choline and polyethylene glycol.

In a still further embodiment, the present invention relates to a stable liquid pharmaceutical composition comprising
a) about 5 mg/mL to about 200 mg/mL of cabazitaxel or a pharmaceutically acceptable salt or solvate thereof, and
b) a solubilizer, wherein the solubilizer is a mixture of phosphatidyl choline and polyethylene glycol, wherein the pharmaceutical composition is substantially free of
   (i) polysorbates and
   (ii) stabilizers selected from the group consisting of preservatives, antioxidants and chelating agents.

In embodiments of the invention the stable liquid pharmaceutical composition of cabazitaxel typically comprise less than 85% w/v of polyethylene glycol. In embodiment of the invention, the formulation shall comprise of about 20% w/v to about 75% w/v, more preferably of about 25% w/v to about 70% w/v of polyethylene glycol and more preferably of about 65% w/v of polyethylene glycol.

In embodiments of the invention the stable liquid pharmaceutical composition of cabazitaxel typically comprise of about 0.1% w/v to about 10% w/v of soya phosphatidyl choline (SPC), more preferably of about 0.2% w/v to about 5% w/v of soya phosphatidyl choline (SPC), even more preferably of about 0.2% w/v to about 3% w/v of soya phosphatidyl choline (SPC) and most preferably of about 1.25% w/v of soya phosphatidyl choline (SPC).

In an embodiment of the invention, the present invention relates to a stable liquid pharmaceutical composition comprising
a) about 5 mg/mL to about 200 mg/mL of cabazitaxel or a pharmaceutically acceptable salt or solvate thereof,
b) about 0.2% w/v to about 3% w/v of soya phosphatidyl choline, and
c) about 25% w/v to about 70% w/v of polyethylene glycol.

In a further embodiment of the invention, the present invention relates to a stable liquid pharmaceutical composition comprising
a) about 5 mg/mL to about 200 mg/mL of cabazitaxel or a pharmaceutically acceptable salt or solvate thereof,
b) about 0.2% w/v to about 3% w/v of soya phosphatidyl choline, and
c) about 25% w/v to about 70% w/v of polyethylene glycol, wherein the pharmaceutical composition is substantially free of
   (i) polysorbates and
   (ii) stabilizers selected from the group consisting of preservatives, antioxidants and chelating agents.

Solvents preferably used in the present invention are pharmaceutically acceptable alcohols, acetone, acetonitrile, chloroform, dichloromethane or mixtures thereof.

Pharmaceutically acceptable alcohols are selected from the group consisting of ethanol, benzyl alcohol, tertiary-butyl alcohol, isopropyl alcohol, and suitable mixtures thereof. Ethanol is the most preferably used solvent.

In embodiments of the invention the stable liquid pharmaceutical composition of cabazitaxel typically comprise of about 5% w/v to about 40% w/v of ethanol, more preferably of about 10% w/v to about 35% w/v of ethanol and most preferably of about 30% w/v of ethanol.

Co-solvents preferably used in the present invention are polyvinylpyrrolidone, polyvinyl alcohol, glycerine or combinations thereof. Polyvinylpyrrolidone is the most preferably used co-solvent.

In embodiments of the invention the stable liquid pharmaceutical composition of cabazitaxel typically comprise of about 0.1% w/v to about 5% w/v of polyvinylpyrrolidone, more preferably of about 1% w/v to about 3% w/v of polyvinylpyrrolidone and most preferably of about 2.5% w/v of polyvinylpyrrolidone.

In another embodiment, the present invention provides a stable liquid pharmaceutical composition comprising
a) cabazitaxel or a pharmaceutically acceptable salt or solvate thereof,
b) one or more solubilizer, wherein solubilizer is selected from group consisting of soya phosphatidyl choline, polyethylene glycol and glycocholic acid,
c) a solvent and
d) a co-solvent.

In a further embodiment, the present invention provides a stable liquid pharmaceutical composition comprising
a) cabazitaxel or a pharmaceutically acceptable salt or solvate thereof,
b) one or more solubilizer, wherein solubilizer is selected from group consisting of soya phosphatidyl choline, polyethylene glycol and glycocholic acid,
c) a solvent and
d) a co-solvent,
   wherein the pharmaceutical composition is substantially free of
   (i) polysorbates and
   (ii) stabilizers selected from the group consisting of preservatives, antioxidants and chelating agents.

In a specific embodiment, the present invention provides a stable liquid pharmaceutical composition comprising
a) cabazitaxel or a pharmaceutically acceptable salt or solvate thereof,
b) soya phosphatidyl choline,
c) polyethylene glycol,
d) glycocholic acid,
e) ethanol and
f) polyvinylpyrrolidone.

In a specific embodiment, the present invention provides a stable liquid pharmaceutical composition comprising
a) cabazitaxel or a pharmaceutically acceptable salt or solvate thereof,
b) soya phosphatidyl choline,
c) polyethylene glycol,
d) glycocholic acid,
e) ethanol and
f) polyvinylpyrrolidone,
   wherein the pharmaceutical composition is substantially free of
   (i) polysorbates and
   (ii) stabilizers selected from the group consisting of preservatives, antioxidants and chelating agents.

In a further embodiment, the present invention provides a stable liquid pharmaceutical composition comprising
a) cabazitaxel or a pharmaceutically acceptable salt or solvate thereof,
b) soya phosphatidyl choline,
c) polyethylene glycol,
d) ethanol and
e) polyvinylpyrrolidone.

In another embodiment, the present invention provides a stable liquid pharmaceutical composition comprising
a) cabazitaxel or a pharmaceutically acceptable salt or solvate thereof,
b) soya phosphatidyl choline,
c) polyethylene glycol,
d) ethanol and
e) polyvinylpyrrolidone,
   wherein the pharmaceutical composition is substantially free of
   (i) polysorbates and
   (ii) stabilizers selected from the group consisting of preservatives, antioxidants and chelating agents.

In embodiments of the invention, the present invention provides a stable liquid pharmaceutical composition comprising
a) about 5 mg/mL to about 200 mg/mL of cabazitaxel or a pharmaceutically acceptable salt or solvate thereof,
b) about 0.2% w/v to about 3% w/v of soya phosphatidyl choline,
c) about 25% w/v to about 70% w/v of polyethylene glycol,
d) about 10% w/v to about 35% w/v of ethanol and
e) about 1% w/v to about 3% w/v of polyvinylpyrrolidone.

In embodiments of the invention, the present invention provides a stable liquid pharmaceutical composition comprising
a) about 5 mg/mL to about 200 mg/mL of cabazitaxel or a pharmaceutically acceptable salt or solvate thereof,
b) about 0.2% w/v to about 3% w/v of soya phosphatidyl choline,
c) about 25% w/v to about 70% w/v of polyethylene glycol,
d) about 10% w/v to about 35% w/v of ethanol and
e) about 1% w/v to about 3% w/v of polyvinylpyrrolidone,
   wherein the pharmaceutical composition is substantially free of
   (i) polysorbates and
   (ii) stabilizers selected from the group consisting of preservatives, antioxidants and chelating agents.

In embodiments of the invention, the present invention provides a stable liquid pharmaceutical composition consisting of
a) about 5 mg/mL to about 200 mg/mL of cabazitaxel or a pharmaceutically acceptable salt or solvate thereof,
b) about 0.2% w/v to about 3% w/v of soya phosphatidyl choline,
c) about 25% w/v to about 70% w/v of polyethylene glycol,
d) about 10% w/v to about 35% w/v of ethanol and
e) about 1% w/v to about 3% w/v of polyvinylpyrrolidone.

In embodiments of the invention, the present invention provides a stable liquid pharmaceutical composition consisting of
a) about 5 mg/mL to about 200 mg/mL of cabazitaxel or a pharmaceutically acceptable salt or solvate thereof,
b) about 0.2% w/v to about 3% w/v of soya phosphatidyl choline,
c) about 25% w/v to about 70% w/v of polyethylene glycol,
d) about 10% w/v to about 35% w/v of ethanol and
e) about 1% w/v to about 3% w/v of polyvinylpyrrolidone,
   wherein the pharmaceutical composition is substantially free of
   (i) polysorbates and
   (ii) stabilizers selected from the group consisting of preservatives, antioxidants and chelating agents.

In another embodiment, the stable, liquid pharmaceutical composition of the present invention is in the form of a nanodispersion comprising nanoparticles having a mean size less than 600 nm, preferably less than 400 nm. In another embodiment, the stable, liquid pharmaceutical composition of the present invention is in the form of a nanodispersion comprising nanoparticles having a mean size less than 600 nm, preferably less than 400 nm.

The particle size of the nanoparticles is determined using conventional methods of measuring and expressing particle size like Malvern particle size analysis, sieving, light scattering optical microscopy, image analysis, sedimentation and such other methods known to one skilled in the art. Particle size distribution information can be obtained from the values D10, D50, D90, and Z-Average such as can be generated from a Malvern particle size determination. Without wishing to be bound by any theory, the applicants believe that the delivery of drug through nanodispersion comprising nanoparticles having mean size less than 600 nm, preferably, less than 400 nm, leads to enhanced internalization and accumulation of the drug in the target tumor tissues and cells. Such increased internalization levels provide a potent treatment strategy for curing tumors associated with prostate cancer.

According to one embodiment of the present invention, the particle size of the nanoparticles is in the range of 10 nm to 600 nm.

In another embodiment of the present invention, the cabazitaxel formulation can from nano-particulate formulation on final dilution.

In another embodiment, the present invention provides a method for preparing a single-container, liquid cabazitaxel formulation in an enclosed container. The liquid cabazitaxel formulation in the single vial is stable and is ready to be diluted once and administered to a patient.

The formulations can be presented in unit-dose or multi-dose sealed containers, such as ampules and vials, and can be stored in a freeze-dried (lyophilized) condition requiring only the addition of the sterile liquid excipient e.g., water for injection, immediately prior to use.

In another embodiment, the present invention relates to a stable liquid pharmaceutical composition for use in the treatment of prostate cancer, comprising
a) cabazitaxel or a pharmaceutically acceptable salt or solvate thereof,
b) soya phosphatidyl choline,
c) polyethylene glycol,
d) ethanol and
e) polyvinylpyrrolidone
   wherein the prostate cancer treatment does not require the dexamethasone premedication.

In a still further embodiment, the present invention relates to a stable liquid pharmaceutical composition for use in the treatment of prostate cancer, comprising
a) about 5 mg/mL to about 200 mg/mL of cabazitaxel or a pharmaceutically acceptable salt or solvate thereof,
b) about 0.2% w/v to about 3% w/v of soya phosphatidyl choline,
c) about 25% w/v to about 70% w/v of polyethylene glycol,
d) about 10% w/v to about 35% w/v of ethanol and
e) about 1% w/v to about 3% w/v of polyvinylpyrrolidone,
   wherein the pharmaceutical composition is substantially free of
   (i) polysorbates and
   (ii) stabilizers selected from the group consisting of preservatives, antioxidants and chelating agents and
   wherein the prostate cancer treatment does not require the dexamethasone premedication.

In another embodiment, the present invention relates to increasing survival comprising administering to a patient in need thereof a liquid pharmaceutical composition comprising
a) cabazitaxel or a pharmaceutically acceptable salt or solvate thereof,
b) soya phosphatidyl choline,
c) polyethylene glycol,
d) ethanol and
e) polyvinylpyrrolidone,
   wherein the administration does not require the dexamethasone premedication and
   wherein said patient has castration resistant metastatic prostate cancer that has progressed during or after treatment with docetaxel.

In still further another embodiment, the present invention relates to increasing survival comprising administering to a patient in need thereof a liquid pharmaceutical composition comprising
a) about 5 mg/mL to about 200 mg/mL of cabazitaxel or a pharmaceutically acceptable salt or solvate thereof,
b) about 0.2% w/v to about 3% w/v of soya phosphatidyl choline,
c) about 25% w/v to about 70% w/v of polyethylene glycol,
d) about 10% w/v to about 35% w/v of ethanol and
e) about 1% w/v to about 3% w/v of polyvinylpyrrolidone, wherein the pharmaceutical composition is substantially free of
   (i) polysorbates and
   (ii) stabilizers selected from the group consisting of preservatives, antioxidants and chelating agents,
   wherein the administration does not require the dexamethasone premedication and
   wherein said patient has castration resistant metastatic prostate cancer that has progressed during or after treatment with docetaxel.

The following examples further illustrate the invention but should not be construed as in any way limiting its scope. In particular, the processing conditions are merely exemplary and can be readily varied by one of ordinary skill in the art.

### Example 1: Cabazitaxel Liquid Formulation 100 mg/mL

To a sterile vessel 1.75 g of Soya Phosphatidyl Choline (S-100) was added and dissolved in 5.25 mL of ethanol. To the above vessel 20 mL of polyethylene glycol 400 (PEG 400), 105 mg of Sodium Glycocholate, 3.5 g cabazitaxel and 1.4875 g of polyvinylpyrrolidone was added and heated to 40° C. using water bath and the volume was finally made to 35 mL using PEG 400 and mixed for 5 minutes which was filtered using 0.22μ filter, filled into vial and purged with nitrogen and stored at room temperature.

### Example 2: Cabazitaxel Liquid Formulation 100 mg/mL

To a sterile vessel 1.75 g of Soya Phosphatidyl Choline (S-100) was added and dissolved in 5.25 mL of ethanol. To the above vessel 17.5 mL of polyethylene glycol 400 (PEG 400), 3.5 g cabazitaxel and 1.4875 g of polyvinylpyrrolidone was added and heated to 40° C. using water bath and the volume was finally made to 35 mL using PEG 400 and mixed for 5 minutes which was filtered using 0.22μ filter, filled into vial and purged with nitrogen and stored at room temperature.

### Example 3: Cabazitaxel Liquid Formulation 10 mg/mL

Composition

| S. No | Ingredients | mg/mL | % w/v |
|---|---|---|---|
| 1. | Cabazitaxel | 10 | 1% |
| 2. | Soya Phosphatidyl | 12.50 | 1.25% |
| | Choline (S-100) | | |
| 3. | Ethanol | 451.75 | 45.17% |
| 4. | Sodium Glycocholate | 0.75 | 0.075% |
| | (Glycocholic acid) | | |
| 5. | Polyvinylpyrrolidone | 25.0 | 2.5% |
| 6. | Polyethylene glycol 400 | 500.0 | 50% |

| | |
|---|---|
| Total | 1000 |

Process for Preparation

To a sterile vessel Soya Phosphatidyl Choline (S-100) was added and dissolved in ethanol. To the above vessel half the quantity of polyethylene glycol 400 (PEG 400), Sodium Glycocholate, cabazitaxel and polyvinylpyrrolidone was added and solubilized. The volume was finally made up with rest of half quantity PEG 400 to final volume and mixed for 5 minutes which was filtered using 0.24 filter, filled into vial and purged with nitrogen and stored at room temperature.

### Example 4: Cabazitaxel Liquid Formulation 10 mg/mL

Composition

| S. No | Ingredients | mg/mL | % w/v |
|---|---|---|---|
| 1. | Cabazitaxel | 10 | 1% |
| 2. | Soya Phosphatidyl | 12.50 | 1.25% |
| | Choline (S-100) | | |
| 3. | Ethanol | 300.0 | 30% |
| 4. | Polyvinylpyrrolidone | 25.0 | 2.5% |
| 5. | Polyethylene glycol 400 | 652.5 | 65.25% |

| | |
|---|---|
| Total | 1000 |

Process for Preparation

To a sterile vessel Soya Phosphatidyl Choline (S-100) was added and dissolved in ethanol. To the above vessel half quantity of polyethylene glycol 400 (PEG 400), cabazitaxel and polyvinylpyrrolidone was added and solubilized. The volume was finally made up with rest half quantity of PEG 400 to final volume and mixed for 5 minutes which was filtered using 0.24 filter, filled into vial and purged with nitrogen and stored at room temperature.

### Example 5: Stability Study

The compositions prepared according to Example 3 and 4 are subjected to stability studies and the results are summarized in below Table—

TABLE 1
Stability Study results at 40° C./75% RH
Impurity Content (in %) w.r.t to time (in days)

| Composition No. | Initial | 8 Days | 15 Days | 30 Days | 60 Days |
|---|---|---|---|---|---|
| Example 3 | 0.19 | Not | Not | 1.04 | 2.083 |
| | | Done | Done | | |
| Example 4 | 0.14 | 0.19 | 0.19 | 0.26 | 0.18 |

### Example 6: Dilution Stability Study

The compositions prepared according to Example 4 are diluted with 5% dextrose solution and 0.9% sodium chloride solution to make the final concertation of 0.1 mg/mL and 0.26 mg/mL of cabazitaxel and are subjected to stability and the results are summarized in below Table-2.

TABLE 2
Stability Study results at Room Temperature
Impurity Content (in %) w.r.t to time (in Hours)

| Composition No. | | 2 | 4 | 6 | 8 | 10 |
|---|---|---|---|---|---|---|
| 4 (Dilution) | Initial | Hour | Hour | Hour | Hour | Hour |
| Dilution with | 0.06 | Not | 0.1 | 0.1 | 0.12 | Not |
| 5% Dextrose | | Done | | | | Done |
| (0.1 mg/mL) | | | | | | |
| Dilution with | 0.14 | 0.15 | 0.13 | 0.16 | 0.16 | 0.14 |
| 5% Dextrose | | | | | | |
| (0.26 mg/mL) | | | | | | |
| Dilution with | Not | 0.15 | 0.11 | Not | 0.14 | 0.15 |
| 0.9% Sodium | Done | | | Done | | |
| Chloride | | | | | | |
| (0.1 mg/mL) | | | | | | |
| Dilution with | 0.15 | 0.16 | 0.17 | 0.13 | 0.15 | 0.17 |
| 0.9% Sodium | | | | | | |
| Chloride | | | | | | |
| (0.26 mg/mL) | | | | | | |

Particle Size of the Diluted Solution

The compositions prepared according to Example 4 are diluted with 5% dextrose solution and 0.9% sodium chloride solution to make the final concertation of 0.1 mg/mL and the particle size of the composition was determined by Malvern Zeta sizer light scattering experiment and the results are tabulated in Table-3.

TABLE 3
Particle Size after Dilution of Example-4 Solution

| Composition | | | | |
|---|---|---|---|---|
| No. 4 (Dilution) | D10 | D50 | D90 | Z-Average |
| Dilution with 5% | 87 | 194 | 442 | 170.5 |
| Dextrose (0.1 mg/mL) | | | | |
| Dilution with 0.9% | 92.9 | 184 | 828 | 213.1 |
| Sodium Chloride | | | | |
| (0.1 mg/mL) | | | | |

We claim:

1. A stable liquid pharmaceutical composition comprising
a) cabazitaxel or a pharmaceutically acceptable salt or solvate thereof,
b) one or more solubilizers selected from the group consisting of soya phosphatidyl choline, polyethylene glycol and glycocholic acid,
c) a solvent and
d) a co-solvent,
wherein the pharmaceutical composition is substantially free of
(i) polysorbates and
(ii) stabilizers selected from the group consisting of preservatives, antioxidants and chelating agents.

2. The stable liquid pharmaceutical composition as claimed in claim 1, wherein the solvent is selected from group consisting of ethanol, benzyl alcohol, tertiary butyl alcohol and isopropyl alcohol.

3. The stable liquid pharmaceutical composition as claimed in claim 1, wherein the co-solvent is selected from the group consisting of polyvinylpyrrolidone, polyvinyl alcohol and glycerin.

4. A stable liquid pharmaceutical composition comprising
a) cabazitaxel or a pharmaceutically acceptable salt or solvate thereof,
b) soya phosphatidyl choline,
c) polyethylene glycol,
d) ethanol and
e) polyvinylpyrrolidone.

5. The stable liquid pharmaceutical composition as claimed in claim 4, wherein the composition comprises of about 5 mg/mL to about 200 mg/mL of cabazitaxel or a pharmaceutically acceptable salt or solvate thereof.

6. The stable liquid pharmaceutical composition as claimed in claim 4, wherein the composition comprises of about 0.2% w/v to about 3% w/v of soya phosphatidyl choline.

7. The stable liquid pharmaceutical composition as claimed in claim 4, wherein the composition comprises of about 25% w/v to about 70% w/v of polyethylene glycol.

8. The stable liquid pharmaceutical composition as claimed in claim 4, wherein the composition comprises of about 10% w/v to about 35% w/v of ethanol.

9. The stable liquid pharmaceutical composition as claimed in claim 4, wherein the composition comprises of about 1% w/v to about 3% w/v of polyvinylpyrrolidone.

10. A stable liquid pharmaceutical composition consisting of
a) about 5 mg/mL to about 200 mg/mL of cabazitaxel or a pharmaceutically acceptable salt or solvate thereof,
b) about 0.2% w/v to about 3% w/v of soya phosphatidyl choline,
c) about 25% w/v to about 70% w/v of polyethylene glycol,
d) about 10% w/v to about 35% w/v of ethanol and
e) about 1% w/v to about 3% w/v of polyvinylpyrrolidone,
wherein the pharmaceutical composition is substantially free of
(i) polysorbates and
(ii) stabilizers selected from the group consisting of preservatives, antioxidants and chelating agents.
